(19) United States
(12) Patent Application Publication
(10) Pub. No.: US 2004/0176321 A1
(43) Pub. Date: Sep. 9, 2004

(54) Compositions of benzoquinolizine carboxylic acid antibiotic drugs

(75) Inventors: Dilip G. Saoji, Aurangabad (IN); Rajendra N. Nagori, Aurangabad (IN); Ravindra D. Yeole, Aurangabad (IN); Nitin Shetty, Aurangabad (IN); Milind C. Shukla, Aurangabad (IN); Noel J. de Souza, Mumbai (IN)

Correspondence Address: Ladas & Parry, 26 West 61 Street, New York, NY, 10023, US

(73) Assignee: WOCKHARDT LIMITED

(21) Appl. No.: 10/749,931

(22) Filed: Dec. 31, 2003

(30) Foreign Application Priority Data
Dec. 31, 2002 (IN) 1169/MUM/2002

(51) Int. Cl. A61K 31/724; A61K 31/4745

(52) U.S. Cl. 514/58; 514/295

(57) ABSTRACT

The present invention relates to a pharmaceutical composition in aqueous solution form useful for parenteral application to a subject for treatment or prevention of infective disease. In particular the present invention relates to such a composition having as an active agent S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or a benzoquinolizine-2-carboxylic acid antibiotic drug. The field of the invention also includes processes for the preparation of such a composition, the use of such a composition in preparation of a medicament, and to the therapeutic or prophylactic use of such a composition.

The present invention relates to a pharmaceutical composition for therapeutic or prophylactic administration to a subject having an infective disease or at risk thereof. The composition comprises an aqueous carrier having in solution therein S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or in general a benzoquinolizine-2-carboxylic acid antimicrobial drug or a polymorphic form, enantiomeric form, other isomeric or racemic form thereof, in a therapeutically or prophylactically effective drug concentration that is above the practical limit of solubility of the drug in a substantially isotonic aqueous solution at a physiologically compatible pH, and a pharmaceutically acceptable solubilising agent, such an agent being a basic amino-acid or a cyclodextrin, or both a basic aminoacid and a cyclodextrin, in a concentration sufficient to maintain the drug in solution at such a drug concentration. The composition is particularly useful for intravenous delivery of the drug, both as ready to use injection and/or infusion solutions and dosage forms which can be converted into such injection and/or infusion solutions before use.

### FIELD OF THE INVENTION

The present invention relates to a pharmaceutical composition in aqueous solution form useful for parenteral application to a subject for treatment or prevention of infective disease. In particular the present invention relates to such a composition having as an active agent S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or a benzoquinolizine-2-carboxylic acid antibiotic drug. The field of the invention also includes processes for the preparation of such a composition, the use of such a composition in preparation of a medicament, and to the therapeutic or prophylactic use of such a composition.

### BACKGROUND OF THE INVENTION

Achiral and chiral benzoquinolizine-2-carboxylic acid compounds have been reported to have therapeutically and/or prophylactically useful antibiotic or antimicrobial, in particular antibacterial, effects. Among such compounds are those illustratively disclosed in the following patents/applications, each of which is individually incorporated herein by reference.

U.S. Pat. No. 4,399,134

U.S. Pat. No. 4,552,879

U.S. Pat. No. 6,514,986

U.S. Pat. No. 6,608,078

U.S. Pat. No. 6,664,267

EP Patent No. 9,081,81

U.S. application Ser. No. 09/566,875

U.S. application Ser. No. 09/640,947

Compounds disclosed in some of the above cited patents and applications include for example the compound 9-fluoro-6,7-dihydro-8-(4-hydroxypiperidin-1-yl)-5-methyl-1-oxo-1H,5H-benzo[i,j]quinolizine-2-carboxylic acid. Compounds of special relevance to this invention which are referred to herein are for instance compounds disclosed in the above cited patents and applications and correspond to S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate and, S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof. S-(−)-9-fluoro-6,7-dihydro-8-(4-hydroxypiperidin-1-yl)-5-methyl-1-oxo-1H,5H-benzo[i,j]quinolizine-2-carboxylic acid has the structure shown in Formula I.

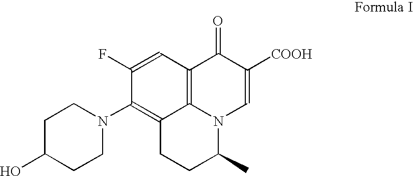

S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate, S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof, and in general appropriately substituted benzoquinolizine-2-carboxylic acids exhibit strong antibacterial activity against sensitive and resistant strains of gram-positive organisms including those of the following genera: Staphylococcus (e.g., Staphylococcus aureus, Staphylococcus epidermidis), Streptococcus (e.g., Streptococcus viridans, Streptococcus pneumoniae), Enterococcus (e.g., Enterococcus faecalis, Enterococcus faecium), anaerobes Bacillus, Corynebacterium Chlamydia and Neisseria, newly-emerging gram-negative organisms such as Chryseobacterium meningosepticum and C. indologense, and gram-negative pathogens such as E.coli, Klebsiella, Proteus, Serratia, Citrobacter and Pseudomonas. The benzoquinolizine-2-carboxylic acid compounds as described in this invention are also generally effective against anaerobic organisms such as those of the genera Bacteroides and Clostridia, and against acid-fast organisms such as those of the genus Mycobacterium such as Mycobacteria tuberculosis, M. intracellulare, M. avium,

S-(−)-9-fluoro-6,7-dihydro-8-(4-hydroxypiperidin-1-yl)-5-methyl-1-oxo-1H,5H-benzo[i,j]quinolizine-2-carboxylic acid, bearing as it does a 4-hydroxypiperidine moiety as an 8-position substituent in the benzoquinolizine-2-carboxylic acid core, has a pKa value of 6.8. It or its 0.2 hydrate does not form, or does not readily form, acid addition salts. In U.S. Pat. No. 4,399,134 and U.S. Pat. No. 4,552,879, it is, however, stated that the described benzoquinolizine-2-carboxylic acid can be converted into a corresponding carboxylate salt with a pharmaceutically acceptable basic compound by using alkali hydroxides and organic bases. The accompanying examples in U.S. Pat. No. 4,399,134 and U.S. Pat. No. 4,552,879 and also EP Patent No. 908181 imply the use of a sodium salt of a benzoquinolizine carboxylic acid without the actual description of its preparation or of its physicochemical properties. The present inventors have shown that S-(−)-9-fluoro-6,7-dihydro-8-(4-hydroxypiperidin-1-yl)-5-methyl-1-oxo-1H,5H-benzo[i,j]quinolizine-2-carboxylic acid and the 0.2 hydrate thereof do, however, form base addition salts with basic amino acids used as counter ions. U.S. Pat. No. 6,514,986 and U.S. Pat. No. 6,664,267 disclose, in particular, the different polymorphic forms of S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof. It is generally difficult to formulate S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-15H-benzo[i,j]quinolizine-2-carboxylic acid arginine salt and polymorphic forms thereof or appropriately substituted benzoquinolizine-2-carboxylic acid drugs as a solution in a pharmaceutically acceptable liquid carrier, particularly in aqueous carrier, in view of their relatively low solubility in water. In the case of RS-(±)-9-fluoro-6,7-dihydro-8-(4-hydroxypiperidin-1-yl)-5-methyl-1-oxo-1H,5H-benzo[i,j]quinolizine-2-carboxylic acid the solubility at ambient temperature is about 0.03 mg/ml. In the case of S-(−)-9-fluoro-6,7-dihydro-8-(4-hydroxypiperidin-1-yl)-5-methyl-1-oxo-11H,5H-benzo[i,j]quinolizine-2-carboxylic acid 0.2 hydrate, for example, the solubility at ambient temperature is less than 0.1 mg/ml. In the case of S-(−)-9-fluoro-6,7-dihydro-8-(4-hydroxypiperidin-1-yl)-5-methyl-1-oxo-1H,5H-benzo[i,j]quinolizine-2-carboxylic acid or S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof, or appropriately substituted benzoquinolizine-2-carboxylic acid drugs for example, the solubility at ambient temperature is less than 1.5 mg/ml.

The above-cited U.S. Pat. Nos. 6,514,986, 6,608,078 and 6,664,267 and U.S. patent applications Ser. Nos. 09/566,875 and 09/640,947 disclose that the subject antibiotic benzoquinolizine-2-carboxylic acids, and in particular S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate and different S-(−)-9-fluoro-6,7-dihydro-8-(4-hydroxypiperidin-1-yl)-5-methyl-1-oxo-1H,5H-benzo[i,j]quinolizine-2-carboxylic acid arginine salts and polymorphic forms thereof, can be formulated as liquid form compositions including solutions. For example, it is disclosed in U.S. Pat. Nos. 6,514,986, 6,608,078 and 6,664,267 and U.S. patent applications Ser. Nos. 09/566,875 and 09/640,947 that the subject benzoquinolizine carboxylic acid compounds can be administered orally, rectally, parenterally, transdermally and/or topically and that parenteral administration can be by intravenous injection, infusion or other parenteral route. For parenteral administration, it is disclosed that a suitable composition will generally contain a pharmaceutically acceptable amount of the subject benzoquinolizine carboxylic acid compound dissolved in a liquid carrier or diluent such as water for injection to form a suitably buffered isotonic solution.

Particularly where parenteral or oral administration of S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or polymorphic forms thereof or appropriately substituted benzoquinolizine-2-carboxylic acid drugs is contemplated, it is desired to achieve systemic concentrations of the drug in the bloodstream above a minimum inhibitory concentration for 90% of a target organism (MIC₉₀). It will readily be understood that it is difficult to achieve such concentrations by administration of a relatively small volume of a composition wherein the drug is present in dissolved form, unless the composition has a relatively high drug concentration, and in particular a drug concentration substantially above the limit of solubility in water of S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or polymorphic forms thereof or in general of most benzoquinolizine-2-carboxylic acids.

A need therefore exists for a solution composition of S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or polymorphic forms thereof or appropriately substituted benzoquinolizine-2-carboxylic acid drug having a drug concentration substantially in excess of the practical limit of solubility of the drug in water. A particular need exists for a parenterally deliverable solution composition of a S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or polymorphic forms thereof or appropriately substituted benzoquinolizine-2-carboxylic acid having a relatively high concentration of the drug.

Some quinolone carboxylic acids are known to cause vein irritation upon infusion and accordingly, adversely affect the use of these compounds for parenteral administration to patients. However, solutions of benzoquinolizine carboxylic acids that reduce vein irritation and even phlebitis and are suitable for administration to human or veterinary patients have not been reported in the literature, and except for Wockhardt's own patent applications, the inventors are not aware of any publication or disclosure of solutions of benzoquinolizine carboxylic acids that reduce vein irritation and even phlebitis and are suitable for administration to human or veterinary patients.

### SUMMARY OF THE INVENTION

The present invention provides a stable pharmaceutical composition suitable for therapeutic or prophylactic administration to a subject having or at risk of infective disease, the composition, ready for use, or before administration converted into a composition of this type, comprising an aqueous carrier having in solution therein (a) a S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or polymorphic forms thereof or appropriately substituted benzoquinolizine-2-carboxylic acid drug in a therapeutically or prophylactically effective drug concentration that is above the practical limit of solubility of the drug in a substantially isotonic aqueous solution at a physiologically compatible pH, and (b) a pharmaceutically acceptable solubilising agent, such an agent being a basic amino-acid or a cyclodextrin or both a basic amino acid and a cyclodextrin, in a concentration sufficient to maintain the drug in solution at such a drug concentration. Preferably the drug concentration is in a range of about 1 mg/ml to about 100 mg/ml of the composition.

The term “stable” in the present context encompasses compositions stable to light under the normal conditions for use and stable to temperature while having a pH compatible with direct administration.

The term “suitable for therapeutic or prophylactic administration” in the present context encompasses compositions such as injection solutions or infusion solutions that are suitable for direct administration as formulated, compositions that are suitable for administration upon dilution in an appropriate pharmaceutically acceptable liquid, and also other presentations which before administration are converted into injection solutions or infusion solutions of this type. The term “infusion solution” in the present context encompasses a pharmaceutical composition obtained by dissolving the drug in water or other aqueous physiologically compatible vehicles to enable drug delivery of the composition through the venous system.

Where the composition is intended for direct administration as formulated, the drug concentration is more preferably about 4 mg/ml to about 12 mg/ml and most preferably about 5 mg/ml to about 9 mg/ml.

Investigation by the inventors of the pH-solubility profile of S-(−)-9-fluoro-6,7-dihydro-8-(4-hydroxypiperidin-1-yl)-5-methyl-1-oxo-1H,5H-benzo[i,j]quinolizine-2-carboxylic acid with different counterions to provide a stable solution dosage form, which would reduce vein irritation and phlebitis and would also comply with safety requirements of drug regulatory authorities, such as abnormal toxicity, led the inventors to the choice of a pharmaceutically acceptable basic amino-acid.

Use by the inventors of counterions other than amino acid like for instance the previously used sodium described in U.S. Pat. No. 4,399,134 and U.S. Pat. No. 4,552,879 and also EP Patent No. 908181 failed in respect of providing a solution with one or more of the following requirements such as being devoid of phlebitogenic properties, free of abnormal toxicity, in remaining sufficiently stable, or for utility as a marketable parenteral drug. The present invention is based in part on the establishment that addition of an amount of amino acid, in particular of the amino acid arginine, in a prescribed range provides to a surprising degree a solution with (a) increased solubility of benzoquinolizine-2-carboxylic acid, (b) lowered potential to induce phlebitogenicity, (c) fulfilling the abnormal toxicity regulatory requirements and (d) stability when stored for an extended period at specified temperature and humidity ranges. These attributes, among other benefits, make it possible for the first time to deliver intravenously a therapeutically or prophylactically effective dose of S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or polymorphic forms thereof or appropriately substituted benzoquinolizine-2-carboxylic acid drug in a volume small enough to be clinically acceptable and convenient, even for subjects intolerant of large volume intravenous infusion because of hypertension, cardiac, renal and/or other problems. For example, a 900 mg dose of S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and/or polymorphic forms thereof can, through use of a composition of the present invention incorporating arginine, be delivered intravenously in a volume of 100 ml or less.

Similar investigations by the inventors also led them to the choice of an appropriate cyclodextrin for enhancing the solubility of the aforementioned drugs. It is believed, without being bound by theory, that the enhanced solubility of the drug in a composition of the invention comprising cyclodextrin is due to association of at least a portion of the drug with the cyclodextrin. It is further believed that at least one mechanism by which the drug associates with the cyclodextrin compound to enhance solubility of the drug in an aqueous medium is through formation of an inclusion complex. The cyclodextrin complex may be formed with the unionized acidic drug itself or with the salt of the acidic drug. Such complexes or conjugates are known in the art to form with a variety of drugs, and a number of advantages have been postulated for use of cyclodextrin-drug complexes in pharmacy. See for example review articles by Bekers et al. (1991) in Drug Development and Industrial Pharmacy, 17, 1503-1549, Szejtli (1994) in Medical Research Reviews, 14, 353-386; Zhang & Rees (1999) in Export Opinion on Therapeutic Patents, 9,1697-1717; and Redenti et al, (2001) in J. Pharm. Sci., 90, 979-986.

Formulations of various drugs with various cyclodextrins have been proposed in the patent literature, including the patents and publications referenced below.

U.S. Pat. No. 5,670,530 discloses compositions comprising a rhodacyanine anti-cancer agent and a cyclodextrin.

U.S. Pat. No. 5,756,546 discloses compositions comprising nimesulide and a cyclodextrin.

U.S. Pat. No. 5,807,895 discloses compositions comprising a prostaglandin and a cyclodextrin.

U.S. Pat. No. 5,824,668 discloses compositions comprising a 5β steroid drug and a cyclodextrin.

International Patent Publication No. WO 96/32135 discloses compositions comprising propofol and a cyclodextrin.

International Patent Publication No. WO 96/38175 discloses compositions comprising an antiulcerative benzimidazole compound and a branched cyclodextrin-carboxylic acid.

International Patent Publication No. WO 97/39770 discloses compositions comprising a thrombin inhibitor and a cyclodextrin.

International Patent Publication No. WO 98/37884 discloses compositions comprising a 3,4-diarylchroman compound and a cyclodextrin.

International Patent Publication No. WO 98/55148 discloses compositions comprising a sparingly water-soluble drug, a cyclodextrin, a water-soluble acid and a water-soluble organic polymer.

International Patent Publication No. WO 98/58677 discloses compositions comprising voriconazole and a cyclodextrin.

International Patent Publication No. WO 99/2073 discloses compositions comprising a taxoid such as paclitaxel or docetaxel and a cyclodextrin.

International Patent Publication No. WO 99/27932 discloses compositions comprising an antifungal compound of defined formula and a cyclodextrin.

International Patent Publication No. WO 01/82971 discloses compositions comprising a glycopeptide antibiotic and a cyclodextrin.

International Patent Publication No. WO 02/15940 discloses compositions comprising an oxazolidinone antimicrobial drug and a cyclodextrin.

International Patent Publication No. WO 02/47660 discloses compositions comprising dronedarone and a cyclodextrin.

Cyclodextrins are expensive excipients and in many cases the degree of enhancement of solubility, or other benefit obtained, has not economically justified the increased cost of a formulation arising from addition of a cyclodextrin. The present invention is based in part on the discovery that addition of a relatively modest amount of cyclodextrin compound increases the solubility of a S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or polymorphic forms thereof or appropriately substituted benzoquinolizine-2-carboxylic acid drug or its salt with a basic amino acid to a surprising degree. For example, a 900 mg dose of S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof can, through use of a composition of the present invention incorporating hydroxypropyl β-cyclodextrin, be delivered intravenously in a volume of 100 ml or less.

The term “pharmaceutically acceptable” in relation to an amino acid or cyclodextrin or other excipient herein means having no persistent detrimental effect on the health of the subject being treated. The pharmaceutical acceptability of an amino acid or cyclodextrin depends, among other factors, on the particular amino acid or cyclodextrin compound in question, on its concentration in the administered composition, and on the route of administration.

The term “practical limit of solubility” in relation to a drug means the highest concentration at which the drug can be formulated in solution without risk of precipitation or crystallization of the drug during the normal range of manufacturing, packaging, storage, handling and use conditions. Typically the practical limit of solubility is considerably lower than the true solubility limit in a given aqueous medium, for example about 70% of the true solubility limit. Thus, illustratively, for a drug having a true solubility limit in a given aqueous medium of 2.9 mg/ml, the practical limit of solubility is likely to be about 2 mg/ml.

Except where the context demands otherwise, use of the singular herein will be understood to embrace the plural. For example, by indicating above that a composition of the invention comprises “a benzoquinolizine-2-carboxylic acid antibiotic drug” and “a pharmaceutically acceptable aminoacid or both a basic aminoacid and a cyclodextrin compound”, it will be understood that the composition can contain one or more such drugs and one or more such aminoacids and/or cyclodextrin compounds. The invention also provides a method of preparing a medicament for treating or preventing infective disease, using a composition as described herein.

Also embraced by the present invention is a method of treating or preventing infective disease in a subject, the method comprising administration to the subject of a composition as described above in a therapeutically or prophylactically effective dose. Such administration can be oral, parenteral or topical, but is preferably parenteral and more preferably by intravenous injection or infusion.

The method of the invention is particularly useful where the infective disease arises through infection by one or more gram-positive bacteria, for example those of the genera Staphylococcus (e.g., Staphylococcus aureus, Staphylococcus epidermidis), Streptococcus (e.g., Streptococcus viridans, Streptococcus pneumoniae), Enterococcus (e.g., Enterococcus faecalis, Enterococcus faecium), Bacillus, Corynebacterium, Chlamydia and Neisseria, anaerobic organisms, for example those of the genera Bacteroides and Clostridia, and acid-fast organisms, for example those of Mycobacterium. The method of the invention is especially useful where infection is by a strain of gram-positive bacteria that is resistant to fluoroquinolone, β-lactam, macrolide, oxazolidinone, streptogramin, and/or lipopeptide antibiotics.

### DETAILED DESCRIPTION OF THE INVENTION

The present invention describes using a benzoquinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate, or S-(−)-9-fluoro-6,7-dihydro-8-(4-hydroxypiperidin-1-yl)-5-methyl-1-oxo-1H,5H-benzo[i,j]quinolizine-2-carboxylic acid or a polymorphic, enantiomeric, isomeric or racemic form thereof, in a composition which can be formulated with an amino acid or cyclodextrin compound or both a basic aminoacid and a cyclodextrin. Other preferred benzoquinolizine-2-carboxylic acid are compounds having Formula-II.

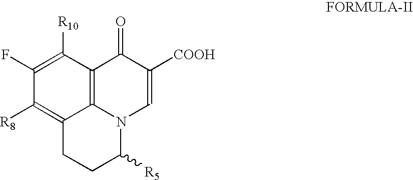

R₅ is C₁₋₆ alkyl, and more preferably R₅ is CH₃, in a stereochemical orientation which is preferably an S-orientation.

R₈ is 4-hydroxypiperidinyl optionally further substituted with one or more C₁₋₆ alkyl, hydroxypiperidinyl optionally further mono/poly substituted with C₁₋₆ alkyl.

More preferably R₈ is

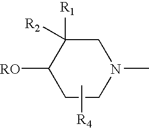

wherein

R is selected from hydrogen, C₁-C₆ alkyl, glycosyl, or aralkyl such as benzyl; or R is C₁-C₆ alkanoyl such as acetyl, propionyl, or pivaloyl, or R is aminoalkanoyl such as an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, or the optically active isomers thereof, or the racemic mixtures thereof, or R is C₆H₁₁O₆, PO₃H₂ or SO₃H thus giving respectively the gluconic acid, phosphoric acid and sulfonic acid ester derivatives of the compounds;

R₁ and R₂ are the same or different and are selected from H, C₁₋₄ alkyl, aralkyl, aminoalkyl, trifluoroalkyl, or halogen;

R₄ is selected from H, C₁₋₄ alkyl, CF₃, phenyl, or F and R₄ is present at one or more of the positions of 2-, 4-, 5-, or 6- of the piperidine ring;

R₁₀ is selected from H, C₁₋₅ alkyl, amino, alkylamino or acylamino; or an optical isomer, diastereomer or enantiomer thereof, or polymorphs, pseudopolymorphs or prodrugs thereof or pharmaceutically acceptable salts and hydrates thereof.

“Optical isomer”, “stereoisomer”, and “diastereomer” as referred to herein have the standard art recognized meanings.

Other examples of preferred benzoquinolizine-2-carboxylic acid of the Formula II are compounds selected from:

S-(−)-9-fluoro-6,7-dihydro-8-(4-hydroxypiperidin-1-yl)-5-methyl-1-oxo-1H,5H-benzo[i,j]quinolizine-2-carboxylic acid or its RS- or R-forms;

S-(−)-9-fluoro-6,7-dihydro-8-(4-hydroxypiperidin-1-yl)-5-methyl-1-oxo-1H,5H-benzo[i,j]quinolizine-2-carboxylic acid arginine salt or polymorphic forms thereof or its RS- or, R-form;

S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate or its RS- or R-form;

S-(−)-9-fluoro-6,7-dihydro-8-{trans-4-(RS)-hydroxy-3-(RS)-methylpiperidin-1-yl}-5-methyl-1-oxo-1H,5H-benzo[i,j]quinolizine-2-carboxylic acid;

S-(−)-9-fluoro-6,7-dihydro-8-{cis-4-(RS)-hydroxy-3-(RS)-methylpiperidin-1-yl-5-methyl-oxo-1H,5H-benzo[i,j]quinolizine-2-carboxylic acid;

S-(−)-9-fluoro-6,7-dihydro-8-{cis-(−)-4-R-hydroxy-3-S-methylpiperidin-1-yl}-5-methyl-1-oxo-1H,5H-benzo[i,j]quinolizine-2-carboxylic acid;

S-(−)-9-fluoro-6,7-dihydro-8-{cis-(+)-4-S-hydroxy-3-R-methylpiperidin-1-yl}-5-methyl-1-oxo-1H,5H-benzo[i,j]quinolizine-2-carboxylic acid; or

S-(−)-9-fluoro-6,7-dihydro-8-(3-ethyl-4-hydroxypiperidin-1-yl)-5-methyl-1-oxo-1H,5H-benzo[i,j]quinolizine-2-carboxylic acid (mixture of cis racemate and trans racemate) or pure stereoisomers thereof.

An embodiment of the invention is that a composition of the invention may include mixtures of optically pure isomers in the ratio of a dextrorotatory form to the levorotatory form of 1%-99%:1%-99%.

The invention is illustrated herein with particular reference to S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof. It will be understood that S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate, any other benzoquinolizine antimicrobial drug can, if desired, be substituted in whole or in part for S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof, with appropriate adjustment in concentration and dosage ranges, in the compositions and methods herein described.

S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof, can be prepared for example, by processes described in the following patents, each of which is individually incorporated herein by reference. U.S. Pat. Nos. 6,514,986 and 6,664,267

S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate and polymorphic forms thereof, can be prepared for example, by processes described in the following patents, each of which is individually incorporated herein by reference. U.S. patent application Ser. No. 09/566,875; WO 00/68229 see Example 2.

Other benzoquinolizine compounds can be prepared by processes known per se, including processes set forth in patent publications disclosing such drugs. U.S. Pat. No. 6,608,078, U.S. Pat. No.4,399,134, U.S. Pat. No.4,552,879 and U.S. application Ser. Nos. 09/566,875 and 09/640,947.

In addition to the active compound, water and other customary formulating auxiliaries, the infusion solutions according to the invention preferably contain an amount, which suffices to dissolve the active compound and to stabilize the solution, of one or more basic amino acid(s) from the group comprising arginine, histidine, arginine acetate, arginine-glutamate, arginine monohydrochloride, histidine acetate, histidine acetate dihydrate, histidine monohydrochloride, histidine monohydrochloride monohydrate, lysine, lysine acetate, lysine monohydrochloride, ornithine, tryptophan, L-arginine, L-histidine, L-arginine acetate, L-arginine-L-glutamate, L-arginine monohydrochloride, L-histidine acetate, L-histidine acetate dihydrate, L-histidine monohydrochloride, L-histidine monohydrochloride monohydrate, L-Lysine, L-Lysine acetate, L-Lysine monohydrochloride and/or a salts thereof and/or D or DL form of these amino acids or salts thereof.

L-arginine and L-lysine or mixtures of L-arginine and L-lysine are particularly preferred.

Preferably, the cyclodextrin is selected from α-cyclodextrin, β-cyclodextrin, γ-cyclodextrin, hydroxypropyl β-cyclodextrin and derivatives thereof. Hydroxypropyl β-cyclodextrin is particularly preferred.

Preferably, the solubilizing agent comprises about 1.5 to about 3.5% by weight of the composition.

When the solubilizing agent is an amino acid, preferably the amino acid comprises about 0.1% to about 1.4% by weight of the composition.

When the solubilizing agent is a cyclodextrin polymer, preferably the cyclodextrin polymer comprises about 1.5% to about 3.5% by weight of the composition.

S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and/or polymorphic forms thereof is usefully present in a composition of the invention at a concentration of about 1 mg/ml to as high a concentration as is practically enabled by the basic amino acid or the cyclodextrin or both the amino acid and the cyclodextrin present therewith, for example about 100 mg/ml. Preferably in a composition intended for direct administration as formulated, the concentration of S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and/or polymorphic forms thereof is about 1 to about 100 mg/ml, more preferably about 4 to about 12 mg/ml, for example about 5 to about 9 mg/ml. Preferably in a composition intended for dilution in a pharmaceutically acceptable liquid prior to administration, the concentration of S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and/or polymorphic forms thereof is about 10 to about 1000 mg/ml, more preferably about 40 to about 120 mg/ml, for example about 50 to about 90 mg/ml. Useful concentrations of other benzoquinolizine drugs are those that are therapeutically equivalent to the S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof and are present in the concentration ranges given immediately above.

Typically, where the composition is intended for direct administration as formulated, suitable concentrations of L-arginine will be found in a range from about 3 to 10 mg/ml, preferably about 5 mg/ml. Where the composition is intended for dilution prior to administration, the concentration of L-arginine will be found in a range of 15 to 25 mg/ml, preferably about 20 mg/ml. Where the composition is a lyophilized product and intended for reconstitution prior to administration, the concentration of L-arginine will be found in a range of 120 to 140 mg/ml, preferably about 130 mg/ml.

Typically, where the composition is intended for direct administration as formulated, suitable concentrations of cyclodextrin will be found in a range from about 15 to 35 mg/ml, preferably about 25 mg/ml. Where the composition is intended for dilution prior to administration, the concentration of cyclodextrin can be significantly higher, for example about 150 to about 350 mg/ml.

One or more pharmaceutically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, citrate/phosphate, sodium bicarbonate and ammonium chloride. Such acids, bases and buffers are included in an amount required to maintain pH of the composition in a physiologically acceptable range, particularly where the composition is intended for parenteral delivery. A physiologically accepted pH range for parenteral delivery is from pH 3 to pH 9.8, preferably pH 5 to pH 9.8.

One or more pharmaceutically acceptable salts or other solutes can be included in the composition in an amount required to bring osmolality of the composition into a physiologically acceptable range, particularly where the composition is intended for parenteral delivery. Such salts include those having sodium, potassium or ammonium cations and chloride, citrate, ascorbate, borate, phosphate, bicarbonate, sulfate, thiosulfate or bisulfite anions; preferred salts include sodium chloride, potassium chloride, sodium thiosulfate, sodium bisulfite and ammonium sulfate, with sodium chloride being especially preferred. A preferred aqueous sodium chloride solution is 0.4-0.9% aqueous sodium chloride. Other solutes suitable for adjustment of osmolality include sugars, for example dextrose, preferably as an aqueous 5% dextrose solution.

Accordingly, a particular embodiment of the invention is a composition as described hereinabove, further comprising a buffering agent and/or an agent for adjusting osmolality in amounts whereby the solution is substantially isotonic and has a physiologically acceptable pH.

Other pharmaceutically acceptable excipients can also be as desired in compositions of the invention, having functions conventional in the art and in amounts consistent with those functions. For example, a water-soluble organic solvent, for example alkaline alcohol, preferably propylene glycol (upto 50%) can be included if desired, as disclosed in U.S. Pat. No. 5,486,508 to Nishida et al., which contemplates a composition suitable for injection comprising a slightly water-soluble drug, a cyclodextrin and a water-soluble organic solvent.

The compositions according to the invention can be prepared by adding and dissolving following ingredients in water or vehicle system: active compound, one or more amino acid(s) or their salts and/or a cyclodextrin intended to ensure complete solubilization of active compound and/or the tonicity regulator and the other adjuvants. The compositions according to the inventions are alternatively prepared by addition of water to a mixture comprising active compound, one or more amino acid(s) or their salts, and/or a cyclodextrin which suffices to dissolve the active compound, and to ensure complete solubilization of active compound, and/or the tonicity regulator and the other adjuvants or else by the addition of active compound and if appropriate other additives such as to a solution of the amino acid(s) and/or a cyclodextrin.

However, the invention also relates to lyophilizates which are prepared by customary techniques such as incorporating an adjuvant, preferably mannitol into the aforesaid described compositions of the invention, and which lyophilizate is converted into the infusion solutions according to the invention by dissolution in solvents suitable for this purpose, for example, conventional infusion vehicle solutions such as water, normal saline or dextrose solution. Lyophilizates of this type can be obtained by freeze-drying of various starting solutions including the active compound, arginine, mannitol and or isotonic agent in aqueous solution, such as, for example, the infusion solutions according to the invention. It is likewise possible to freeze-dry considerably more dilute solutions of active compound-concentration 1 mg/ml as well as considerably more concentrated solutions of active compound-concentration 50 mg/ml than the described infusion solutions in the examples of concentration 9 mg/ml.

The lyophilizates can be prepared both by freeze-drying in the final container such as, for example, in a bottle or ampoule made of glass or plastic, and by bulk freeze-drying combined with dispensing the lyophilizate into a container suitable for this purpose, which takes place at a later time.

The dissolution of the lyophilizate before the administration can be brought about both by addition of a solution, which is suitable for this purpose, into the container containing the lyophilizate, or by addition of the lyophilizate to a suitable solution, or by a combination of procedures of these types.

The composition of the lyophilizates can likewise vary very widely, depending on the composition of the solution which is used for the dissolution.

It can vary from pure active compound to a lyophilizate which contains all the constituents which are to be administered, apart from water.

The invention likewise relates compound to a lyophilizate with solutions containing active compound, which are converted into the infusion solutions according to the invention before the administration.

The invention also includes, powder for reconstitution which have been prepared by customary techniques and which are converted into the infusion solutions according to the invention by dissolution in solvents suitable for this purpose-such as, for example, conventional infusion vehicle solutions.

The powder for reconstitution can be prepared by blending active compound which has been recrystallised in advance under an aseptic condition, in an aseptic environment, with additives like one or more amino acid(s) and/or cyclodextrins and/or isotonicizing agents, as listed above, which have been sterilized separately earlier and the blend is filled in suitable container to obtain active compound solution after reconstitution with vehicle or solvent.

The dissolution of the powder for reconstitution before the administration can be brought about both by addition of a solution which is suitable for this purpose, for example water or an aqueous arginine solution into the container containing the powder and by addition of the powder to a suitable solution, or by a combination of procedures of these types.

The composition of the powder for reconstitution can likewise vary very widely, depending on the composition of the solution which is used for the dissolution.

It can vary from pure active compound to a powder for reconstitution which contain all the constituents which are to be administered, apart from water.

The invention likewise relates to combinations of powder for reconstitution with solutions containing active compound, which are converted into the infusion solutions according to the invention before the administration.

The invention also includes concentrates/suspensions by adding to organic solvents like alkaline glycol, preferably propylene glycol containing dissolved auxiliaries, preferably polysorbate-80, the active compound, arginine and appropriate amounts of water, which are converted into the solutions according to the invention before the administration.

It is possible in this context for these concentrates and suspensions to have various compositions. One possibility would be that which requires merely the addition of water for dilution or dissolution in order to prepare the infusion solutions according to the invention.

The invention also related to other presentations or combinations of presentations which finally result in the infusion solutions according to the invention-and this irrespective of the procedure.

The container into which lyophilizates, concentrates and other presentations such as, for example, suspensions, are dispensed can consist both of glass and of plastic. In this connection, the container materials can contain substances which confer a particular protection on the contents, such as, for example, a protection from light or a protection from oxygen.

Compositions of the present invention can also be prepared by processes known in the art to make compositions for oral, parenteral or topical administration. A process to prepare compositions of this invention includes simple admixture, with agitation as appropriate, of the hereinbefore defined benzoquinolizine-2-carboxylic acid, hydrate, salts, polymorphs, and/or isomers thereof with an amino acid and other adjuvant. A second process involves preparation first of an aqueous solution of the cyclodextrin compound to which is added the hereinbefore defined benzoquinolizine-2-carboxylic acid, hydrate, salts, polymorphs, and/or isomers thereof in finely divided solid particulate form with agitation until it is fully dissolved. Where it is desired to prepare a buffered isotonic solution, for example for intravenous infusion, buffering agents and agents for adjustment of osmolality as herein before defined can be added at any stage but are preferably present in solution with the cyclodextrin compound before addition of the hereinbefore defined benzoquinolizine-2-carboxylic acid, hydrate, salts, polymorphs, isomers thereof. Processes for preparing a composition of the invention, particularly one intended for parenteral use, are preferably conducted so as to provide a sterile product.

Compositions of the invention intended for parenteral administration are generally suitable for packaging and dispensing in conventional intravenous delivery bags and apparatus.

A contemplated composition can be dried, for example by spray drying, to form a reconstitutable powder. The powder can be dissolved in sterile water to reconstitute a parenterally deliverable composition as herein described.

In a method of the invention for treating or preventing infective disease, a composition as described above in a therapeutically or prophylactically effective daily dose is administered to a subject in need thereof. Such administration can be oral, parenteral or topical, but is preferably parenteral and more preferably by intravenous injection or infusion.

In a particular embodiment of the invention, a method for treating or preventing infective disease comprises (a) using a composition of the invention for direct administration (b) diluting a composition as described herein in a pharmaceutically acceptable liquid to form a diluted composition suitable for direct administration, and (c) administering the diluted composition in a therapeutically or prophylactically effective daily dose to a subject in need thereof. Preferably such administration is parenteral and the liquid in which the composition is diluted is a parenterally acceptable aqueous carrier, for example saline or a substantially isotonic buffered aqueous solution, preferably normal saline and/or dextrose solution having a physiologically compatible pH value of 3.0-9.8, preferably a pH of 5.0-8.0.

As indicated above, a method of the invention is particularly useful where the infective disease arise through infection by one or more gram-positive bacteria. Where broader-spectrum antibacterial activity, extending to gram-negative bacteria, is required, a second antimicrobial drug can be administered in co-therapy, including for example coformulation, with the present composition. The second antimicrobial drug is selected to be effective against target gram-negative bacteria. Such co-therapy and coformulation are embodiments of the present invention.

The second antimicrobial drug can illustratively be selected from aminoglycosides, cephalosporins, diaminopyridines, oxazolidinones, sulfonamides and tetracyclines. Among particular antimicrobial drugs of these and other classes, each of the following may illustratively be useful as the second antimicrobial drug according to an embodiment of the present invention: amikacin, cefixime, cefoperazone, cefotaxime, ceftazidime, ceftizoxime, ceftriaxone, imipenem, meropenem, eltapenem, chloramphenicol, clindamycin, colistin, daptomycin, domeclocycline, dexycycline, gentamicin, linezolid, mafenide, methacycline, minocycline, neomycin, oxyteracycline, polymyxin B, pyrimethamine, quinupristin-dalfopristin, silver sulfadiazine, sulfacetamide, sulfisoxazole, tetracycline, tobramycin or trimethoprim.

The present invention also encompasses therapeutic and prophylactic methods involving administration of an antibacterial composition as described herein in co-therapy, including for example coformulation, with one or more drugs other than antibacterial drugs.

Therapeutic and prophylactic methods of the invention are useful for any subject in need thereof. The subject is preferably warm-blooded, more preferably mammalian, and most preferably human. However, a particular embodiment of the invention is a veterinary method of treating a non-human subject, for example a domestic, farm or zoo animal, having or at risk of infective disease, with a composition of the invention.

An appropriate dosage, frequently and duration of administration, i.e. treatment regimen, to be used in any particular situation will be readily determined by one of skill in the art without undue experimentation, and will depend, among other factors, on the particular benzoquinolizine compound(s) present in the composition, on the particular infective disease or condition to be treated or prevented, on the age, weight and general physical condition of the subject, and on other medication being administered to the subject. It is preferred that response to treatment according to the present method be monitored and the treatment regimen be adjusted if necessary in light of such monitoring. Where the benzoquinolizine-2-carboxylic acid is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof, a daily dose for a human subject will generally be about 0.01 mg to 100 mg/kg/day, preferably 0.1-50 mg/kg/day. For an average 70 kg human, this would amount to 0.7 mg to 7 mg/day or preferably 0.7 mg-3.5 mg/day of S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof, administered as a single or divided dosage in a composition of the invention. For other S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate and other benzoquinolizine-2-carboxylic acid as defined herein a daily dose that is therapeutically equivalent to the above dose ranges for S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof can be administered.

### EXAMPLES

The following examples are provided for the purpose of illustrating the present invention but are not to be construed as limiting.

### Test Example 1

### Solubility Study with Arginine

A study was conducted to examine the solubility of S-(−)-9-fluoro-6,7-dihydro-8-(4-hydroxypiperidin-1-yl)-5-methyl-1-oxo-1H,5H-benzo[I,j]quinolizine-2-carboxylic acid and S-(−)-9-fluoro-6,7-dihydro-8-(4-hydroxypiperidin-1-yl)-5-methyl-1-oxo-1H,5H-benzo[I,j]quinolizine-2-carboxylic acid arginine salt in an aqueous system containing different concentrations of arginine.

### Experimental Procedure

Aqueous solutions of L-arginine at concentrations of 5, 10, 15, 25, 50, 100 and 200 mg/ml were prepared. 3 ml of each of these solutions was added to an accurately weighed amount of S-(−)-9-fluoro-6,7-dihydro-8-(4-hydroxypiperidin-1-yl)-5-methyl-1-oxo-1H,5H-benzo[I,j]quinolizine-2-carboxylic acid (Subs. “A”) and separately of S-(−)-9-fluoro-6,7-dihydro-8-(4-hydroxypiperidin-1-yl)-5-methyl-1-oxo-1H,5H-benzo[I,j]quinolizine-2-carboxylic acid arginine salt (Subs. “B”). The flasks were kept ca. 16 hours on a mechanical shaker, maintained at 27° C. and 140 rpm. The solutions were filtered through 0.2 micron syringe filter. The filtrates were diluted appropriately and injected on HPLC. The amounts dissolved were determined by comparing the sample peak area with peak area of standard solution.

The amount of Subs. “A” and Subs. “B” dissolved at each concentration of arginine is shown in the following table:

| Conc. Of Arginine | Solubility of Subs. “A” | Solubility of Subs. “B” |
|---|---|---|
| (mg/ml) | (mg/ml) | (mg/ml) |
| 5 | 5.98 | 50.83 |
| 10 | 11.49 | 56.2 |
| 25 | 27.65 | 62.14 |
| 50 | 60.91 | 73.8 |
| 100 | — | 94.95 |
| 200 | — | 142.86 |

Similarly the solubility of S-(−)-9-fluoro-6,7-dihydro-8-(4-hydroxypiperidin-1-yl)-5-methyl-1-oxo-1H,5H-benzo[I,j]quinolizine-2-carboxylic acid 0.2 hydrate in solutions of different concentrations of arginine was determined.

Test Example 2

### Solubility Study with β-Cyclodextrin and Hydroxypropyl β-Cyclodextrin

A study was conducted to examine the solubility of S-(−)-9-fluoro-6,7-dihydro-8-(4-hydroxypiperidin-1-yl)-5-methyl-1-oxo-1H,5H-benzo[I,j]quinolizine-2-carboxylic acid in an aqueous system containing β-cyclodextrin (β-CD) or hydroxypropyl β-cyclodextrin (HP-β-CD)

Aqueous solutions of (β-CD) or (HP-β-CD) at concentrations of 0, 2, 5, 10 and 50 mg/ml were prepared. 1 ml of each of these solutions was added to an accurately weighed amount (about 20 mg) of S-(−)-9-fluoro-6,7-dihydro-8-(4-hydroxypiperidin-1-yl)-5-methyl-1-oxo-1H,5H-benzo[I,j]quinolizine-2-carboxylic acid (Subs. “A”). The flasks were kept ca. 24 hours on a mechanical shaker, maintained at 27° C. and 140 rpm. The solutions were filtered through 0.2 micron syringe filter. The filtrates were diluted appropriately and injected on HPLC. The amounts dissolved were determined by comparing the sample peak area with peak area of standard solution.

The amount of Subs. “A” dissolved at each concentration of (β-CD) or (HP-β-CD) is shown in the following table:

### Substance A

S-(−)-9-fluoro-6,7-dihydro-8-(4-hydroxypiperidin-1-yl)-5-methyl-1-oxo-1H,5H-benzo[I,j]quinolizine-2-carboxylic acid

### Substance C

RS-(±)-9-fluoro-6,7-dihydro-8-(4-hydroxypiperidin-1-yl)-5-methyl-1-oxo-1H,5H-benzo[I,j]quinolizine-2-carboxylic acid

### Substance D

R-(+)-9-fluoro-6,7-dihydro-8-(4-hydroxypiperidin-1-yl)-5-methyl-1-oxo-1H,5H-benzo[I,j]quinolizine-2-carboxylic acid

| β-CD or | Substance A | | Substance C | | Substance D | |
|---|---|---|---|---|---|---|
| HP-β-CD | mg/ml with | | mg/ml with | | mg/ml with | |
| mg/ml | β-CD | HP-β-CD | β-CD | HP-β-CD | β-CD | HP-β-CD |
| 0 | 0.04 | 0.04 | 0.03 | 0.03 | 0.15 | 0.15 |
| 2 | 0.12 | 0.21 | — | 0.05 | — | 0.37 |
| 5 | 0.27 | 0.28 | 0.22 | 0.11 | 0.71 | 0.50 |
| 10 | 0.53 | 0.55 | 0.40 | 0.31 | 1.43 | 1.08 |
| 50 | — | 2.67 | — | 1.03 | — | 5.01 |

### Test Example 3

### Solubility of S-(−)-9-fluoro-6,7-dihydro-8-(4-hydroxypiperidin-1-yl)-5-methyl-1-oxo-1H,5H-benzo[i,j]quinolizine-2-carboxylic acid arginine salt with hydroxypropyl β-cyclodextrin (HP-β-CD)

Aqueous solutions of (HP-β-CD) at concentrations of 25, 60, 100 and 250 mg/ml were prepared. 1 ml of each of these solutions was added to 10 mg, 25 mg, 40 mg and 90 mg S-(−)-9-fluoro-6,7-dihydro-8-(4-hydroxypiperidin-1-yl)-5-methyl-1-oxo-1H,5H-benzo[i,j]quinolizine-2-carboxylic acid arginine salt accurately weighed. The mixtures were shaken for about one minute to get clear solution. All the solutions were clear indicating full solubility of S-(−)-9-fluoro-6,7-dihydro-8-(4-hydroxypiperidin-1-yl)-5-methyl-1-oxo-1H,5H-benzo[i,j]quinolizine-2-carboxylic acid arginine salt. These solutions were filtered through 0.2 μm Whatman nylon filter. The filtered solutions were tightly covered with polyfilm. The test tubes containing the solutions were mounted on a stand and kept on a mechanical shaker at 150 rpm for 6 hours and then upto 24 hours without shaking.

All solutions remained clear at 24 hrs. The pH of the solutions after 24 hours is shown in the table.

| | Substance B | |
|---|---|---|
| HP-β-CD (mg/ml) | (mg/ml) | pH at 24 hrs. |
| 25.0 | 10 | 7.62 |
| 60.0 | 25 | 7.75 |
| 100.0 | 40 | 7.82 |
| 250.0 | 90 | 7.97 |

### Test Example 4

### Abnormal Toxicity Study

Regulatory References: The study is designed to meet the recommendations of Indian Pharmacopoeia (IP) 1996, Appendix 2.2, Method B, Biological tests and determination, Test for abnormal toxicity; Government of India, Health and Family Welfare.

### Dose Formulation

The composition is administered ‘as such’ at the dose of 120 mg/kg to individual mouse.

Test System And Management: Ten healthy (5 male and 5 female) Swiss mice, approximately 5-6 week old and weighing around 28-30 g, are placed at random in polypropylene cages, each cage containing 5 mice of the same sex. Throughout the experimental period animal room temperature and relative humidity is maintained between 22° C. □ 3° C. and 30 to 70% RH respectively. Illumination is controlled to give 12 hours light and 12 hours dark cycles (8.00 a.m. to 8.00 p.m.) each day. All mice have free access to Ultra-guard water (sterilised and cooled), and autoclaved standard pelleted laboratory animal diet. Autoclaved paddy husk is used as bedding and changed every alternate day.

Prior to final assignment to the study, all Swiss mice are subjected to veterinary examination and those in good state of health are selected.

Experimental Procedure:

Administration of test substance: Swiss mice are administered with the provided composition injection as described in example No.4 as a single intravenous dose. The composition is a clear solution of the test compound at a concentration of 9 mg/ml. The composition was administered intravenously ‘as such’ via tail vein of each mouse with the help of graduated 1 ml disposable syringe fitted with 26½ G needle. Each mouse is given a volume calculated on the basis 120 mg/kg against respective body weight recorded prior to study initiation.

Observations made on the animals: Clinical signs, Body Weight, Mortality

The test formulation passes the abnormal toxicity study as treated animals do not exhibit behavioural changes, mortality and decrease in body weight gain during the seven day observation period following the administration.

### Test Example 5

### Superior Tolerability in Animals

A superior tolerability in animals of the use of composition(s) of the invention based on S-(−)-9-fluoro-6,7-dihydro-8-(4-hydroxypiperidin-1-yl)-5-methyl-1-oxo-1H,5H-benzo[i,j]quinolizine-2-carboxylic acid arginine salt as against the composition based on S-(−)-9-fluoro-6,7-dihydro-8-(4-hydroxypiperidin-1-yl)-5-methyl-1-oxo-1H,5H-benzo[i,j]quinolizine-2-carboxylic acid sodium salt was demonstrated by conducting experiments involving daily repeated i.v. administration of test compositions in rat for a period ranging from 7 days to 28 days.

### Experimental Procedure

### Test Compositions of Arginine Salt

S-(−)-9-fluoro-6,7-dihydro-8-(4-hydroxypiperidin-1-yl)-5-methyl-1-oxo-1H,5H-benzo[i,j]quinolizine-2-carboxylic acid arginine salt was dissolved at a concentration of 75 mg/ml in 27 mg/ml arginine solution.

Test Compositions of Sodium Salt

S-(−)-9-fluoro-6,7-dihydro-8-(4-hydroxypiperidin-1-yl)-5-methyl-1-oxo-1H,5H-benzo[i,j]quinolizine-2-carboxylic acid sodium salt was dissolved at a concentration of 75 mg/ml in distilled water adjusted to pH 9.0.

Wistar rats of body weight range 90-100 gm were treated with i.v. administered above test compositions in doses specified in the table below for a period of 7 days to 28 days. The test doses were administered by injecting 0.5 ml-1.0 ml of test compositions via tail vein of each rat with the help of graduated 1 ml disposable syringe fitted with 26½ G needle. Each repeat dose was administered in 5 male and 5 female rats. Animals were monitored for the induction of phlebitis or its progression to complete venous blockade.

| Test Composition | | No. of |
|---|---|---|
| based on | Dose | phlebitis free days |
| Arginine Salt | 450 mg/kg | 23 |
| Sodium Salt | 300 mg/kg | 8 |

The superior venous tolerability of the composition based on the arginine salt affords it a clinically desirable feature of suitability for repeated long term i.v. administration.

### Test Example 6

### Stability Study

The compositions for injection obtained in Examples 1 & 2 are stored in a constant temperature incubator at 40° C. for 6 months and are observed for physical clarity of solutions. The solutions were found to be clear at the end of the stipulated period.

### Example 1

Preparation of a solution containing S-(−)-9-fluoro-6,7-dihydro-8-(4-hydroxypiperidin-1-yl)-5-methyl-1-oxo-1H,5H-benzo[i,j]quinolizine-2-carboxylic acid arginine salt.

To 80 ml of water for injection, previously rendered inert with nitrogen gas sparging, is added and dissolved 1.0 g L-arginine, 0.9 g S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, and the volume made up to 100 ml with water for injection. The solution thus obtained is filtered through membrane filters, filled in bottles and sterilised in an autoclave at 121° C. for 20 minutes. The pH of the solution is 9.37±0.25.

In the abnormal toxicity study, the solution is found to comply with the requirements.

The solution remains clear after keeping for 6 months at 40° C. without alteration of the pH value.

The solution reduced vein irritation and also blocked any progression to cause severe phlebitis in contrast to the solution prepared from the corresponding sodium salt of S-(−)-9-fluoro-6,7-dihydro-8-(4-hydroxypiperidin-1-yl)-5-methyl-1-oxo-1H,5-benzo[i,j]quinolizine-2-carboxylic acid

### Example 2

Preparation of a solution containing S-(−)-9-fluoro-6,7-dihydro-8-(4-hydroxypiperidin-1-yl)-5-methyl-1-oxo-1H,5H-benzo[i,j]quinolizine-2-carboxylic acid.

The solution was prepared similarly to the process described in Example 1 by replacing S-(−)-9-fluoro-6,7-dihydro-8-(4-hydroxypiperidin-1-yl)-5-methyl-1-oxo-1H,5H-benzo[i,j]quinolizine-2-carboxylic acid arginine salt with an equimolar amount of S-(−)-9-fluoro-6,7-dihydro-8-(4-hydroxypiperidin-1-yl)-5-methyl-1-oxo-1H,5H-benzo[i,j]quinolizine-2-carboxylic acid.

### Example 3

Preparation of a solution containing S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate.

The solution was prepared similarly to the process described in Example 1 by replacing S-(−)-9-fluoro-6,7-dihydro-8-(4-hydroxypiperidin-1-yl)-5-methyl-1-oxo-1H,5H-benzo[i,j]quinolizine-2-carboxylic acid arginine salt with an equimolar amount of S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate.

### Example 4

Preparation of a solution containing S-(−)-9-fluoro-6,7-dihydro-8-(4-hydroxypiperidin-1-yl)-5-methyl-1-oxo-1H,5H-benzo[i,j]quinolizine-2-carboxylic acid arginine salt rendered isotonic with sodium chloride.

To 80 ml of water for injection, previously rendered inert with nitrogen gas sparging, is added and dissolved 1.0 g L-arginine, 0.9 g S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, 0.67 g sodium chloride and the volume made up to 100 ml with water for injection. The solution thus obtained is filtered through membrane filters, filled in bottles and sterilised in an autoclave at 121° C. for 20 minutes. The pH of the solution is 9.75.

In the abnormal toxicity study the solution has found to comply with the requirements.

The solution remains clear after keeping for 6 months at 40° C. without the pH being modified.

The solution reduced vein irritation and also blocked any progression to cause severe phlebitis in contrast to the solution prepared from the corresponding sodium salt of S-(−)-9-fluoro-6,7-dihydro-8-(4-hydroxypiperidin-1-yl)-5-methyl-1-oxo-1H,5H-benzo[i,j]quinolizine-2-carboxylic acid

### Examples 5-6

Solutions similar to the one described in Example 4 were made containing equimolar quantities of S-(−)-9-fluoro-6,7-dihydro-8-(4-hydroxypiperidin-1-yl)-5-methyl-1-oxo-1H,5H-benzo[i,j]quinolizine-2-carboxylic acid and S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate.

### Examples 7, 8, 9, and 10

Preparations of solutions containing S-(−)-9-fluoro-6,7-dihydro-8-(4-hydroxypiperidin-1-yl)-5-methyl-1-oxo-1H,5H-benzo[i,j]quinolizine-2-carboxylic acid arginine salt with various concentrations of L-arginine.

According to the conventional method of manufacturing as described in Example 1 aqueous solutions for injections having the following formulations were prepared.

| | Example | | | |
|---|---|---|---|---|
| Ingredients | 7 | 8 | 9 | 10 |
| S-(−)-9-fluoro-6,7-dihydro-8-(4- | 0.9 g | 0.9 g | 0.9 g | 0.9 g |
| hydroxypiperidin-1-yl)- | | | | |
| 5-methyl-1-oxo-1H, | | | | |
| 5H-benzo [i,j] | | | | |
| quinolizine-2-carboxylic | | | | |
| acid arginine salt | | | | |
| L-arginine | 0.30 g | 0.375 g | 0.45 g | 0.60 g |
| water for injection | q.s. to | q.s. to | q.s. to | q.s. to |
| | 100 ml | 100 ml | 100 ml | 100 ml |
| pH | 9.04 | 9.06 | 9.19 | 9.28 |
| Clarity | Clear | Clear | Clear | Clear |

### Examples 11 and 12

Solutions similar to the ones described in Examples 7-10 were made containing equimolar quantities of S-(−)-9-fluoro-6,7-dihydro-8-(4-hydroxypiperidin-1-yl)-5-methyl-1-oxo-1H,5H-benzo[i,j]quinolizine-2-carboxylic acid and S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate.

### Example 13

Preparation of solution containing S-(−)-9-fluoro-6,7-dihydro-8-(4-hydroxypiperidin-1-yl)-5-methyl-1-oxo-1H,5H-benzo[i,j]quinolizine-2-carboxylic acid arginine salt with hydroxypropyl β-cyclodextrin and rendered isotonic with sodium chloride.

To 90 ml of water for injection, previously rendered inert with nitrogen gas sparging, was added and dissolved 2.50 g hydroxypropyl β-cyclodextrin, 0.9 g S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, 0.80 g sodium chloride and the volume made upto 100 ml with water for injection. The solution thus obtained was filtered through membrane filters, filled in bottles and sterilized in an autoclave at 121° C. for 20 minutes.

The pH of the solution was 7.89±0.25.

The clarity of the solution was clear.

### Example 14

Preparation of solution containing RS-(±)-9-fluoro-6,7-dihydro-8-(4-hydroxypiperidin-1-yl)-5-methyl-1-oxo-1H,5H-benzo[i,j]quinolizine-2-carboxylic acid arginine salt with hydroxypropyl β-cyclodextrin and rendered isotonic with sodium chloride.

To 90 ml of water for injection, previously rendered inert with nitrogen gas sparging, was added and dissolved 0.293 g arginine, 6.0 g hydroxypropyl β-cyclodextrin, 0.6 g RS-(±)-9-fluoro-6,7-dihydro-8-(4-hydroxypiperidin-1-yl)-5-methyl-1-oxo-1H,5H-benzo[i,j]quinolizine-2-carboxylic acid arginine salt and 0.70 g sodium chloride. The volume was made upto 100 ml with water for injection. The solution thus obtained was filtered through membrane filters, filled in bottles and sterilized in an autoclave at 121° C. for 20 minutes.

The pH of the solution was 7.60±0.25.

The clarity of the solution was clear.

### Example 15-16

Solutions similar to the ones described in Example 13 were made containing equimolar quantities of S-(−)-9-fluoro-6,7-dihydro-8-(4-hydroxypiperidin-1-yl)-5-methyl-1-oxo-1H,5H-benzo[i,j]quinolizine-2-carboxylic acid and S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate and using the respective requisite amounts of arginine, hydroxypropyl β-cyclodextrin and sodium chloride.

### Example 17

Preparation of concentrated solution for injection

Preparation of a solution containing S-(−)-9-fluoro-6,7-dihydro-8-(4-hydroxypiperidin-1-yl)-5-methyl-1-oxo-1H,5H-benzo[i,j]quinolizine-2-carboxylic acid arginine salt according to the method used for a concentrate solution as described below for injection which can be used on further dilution with compatible intravenous fluids as described below.

To 5 ml propylene glycol previously rendered inert with nitrogen gas sparging is added and dissolved 2.0 g polysorbate-80, 0.2 g L-arginine, 0.9 g S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, and the volume made up to 10 ml with water for injection. The solution thus obtained is filtered through membrane filters and filled in bottles. The pH of solution is 8.90±0.25.

The solution remains clear after keeping for 6 months at 40° C. without the pH changing value.

### Example 18

### Preparation of Lyophillised Formulation

S-(−)-9-fluoro-6,7-dihydro-8-(4-hydroxypiperidin-1-yl)-5-methyl-1-oxo-1H,5H-benzo[i,j]quinolizine-2-carboxylic acid arginine salt (9% w/v), L-arginine (1 3% w/v) and mannitol (4% w/v) are dissolved in water for injection. After sterilisation filtration the solution is dispensed into vials, 10 ml each and then freeze-dried by a conventional method to obtain a freeze-dried preparation.

The preparation is reconstituted with 10 ml water for injection. The resulting solution is - - - clear.

### Example 19

### Preparation of Powder Formulation

S-(−)-9-fluoro-6,7-dihydro-8-(4-hydroxypiperidin-1-yl)-5-methyl-1-oxo-1H,5H-benzo[i,j]quinolizine-2-carboxylic acid arginine salt (40.91% w/w) & L-arginine (59.09% w/w) are mixed & then aseptically filled with 2.2 g powder mixture in each of 10 ml vials.

The preparation is reconstituted with 10 ml water for injection. The resulting solution is clear.

### Example 20

Compositions similar to all of the Examples 1-19 using a mixture of S-(−)-9-fluoro-6,7-dihydro-8-(4-hydroxypiperidin-1-yl)-5-methyl-1-oxo-1H,5H-benzo[i,j]quinolizine-2-carboxylic acid or 0.2 hydrates or arginine salt thereof with R-(−)-9-fluoro-6,7-dihydro-8-(4-hydroxypiperidin-1-yl)-5-methyl-1-oxo-1H,5H-benzo[i,j]quinolizine-2-carboxylic acid or hydrate or arginine salt thereof in proportions ranging from 99% S-(−)-enantiomer +1% R-(+)-enantiomer to 1% S-(−)-enantiomer +99% R-(−)-enantiomer can be similarly prepared.

1. A pharmaceutical composition for therapeutic or prophylactic administration to a subject having an infective disease or at risk for contracting an infective disease, the composition comprising an aqueous carrier having in solution therein (a) a benzoquinolizine-2-carboxylic acid antimicrobial drug or salt, polymorphic form, enantiomeric form, other isomeric or racemic form thereof in a therapeutically or prophylactically effective drug concentration that is above the practical limit of solubility of the drug in a substantially isotonic aqueous solution at a physiologically compatible pH, and (b) a pharmaceutically acceptable solubilising agent selected from a basic amino-acid, a cyclodextrin, a cyclodextrin polymer or derivative theref or a mixture thereof in a concentration sufficient to maintain the drug in solution at drug concentration that is above the practical limit of solubility of the drug in a substantially isotonic aqueous solution at a physiologically compatible pH.

2. The composition of claim 1, that is suitable for parenteral administration.

3. The composition of claim 1, that is suitable for intravenous injection or infusion.

4. The composition of claim 1, wherein the concentration of a drug is about 1 mg/ml to about 100 mg/ml.

5. The composition of claim 1, wherein the concentration of a drug is about 4 mg/ml to about 12 mg/ml.

6. The composition of claim 1, wherein the concentration of a drug is about 5 mg/ml to about 9 mg/ml.

7. The composition of claim 1, wherein the benzoquinolizine-2-carboxylic acid antimicrobial drug is selected from a compound of the formula: 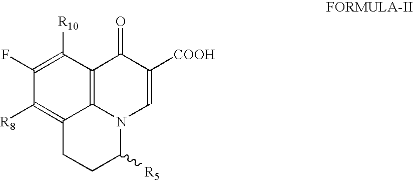
wherein:
R₅ is C₁₋₆ alkyl, as a mixture of enantiomers or in a stereochemical orientation;
R₈ is 4-hydroxypiperidinyl optionally further substituted with one or more C₁₋₆ alkyl, hydroxypiperidinyl optionally further mono/poly substituted with C₁₋₆ alkyl;
R₁₀ is selected from H, C₁₋₅ alkyl, amino, alkylamino or acylamino group; or an optical isomer, diastereomer or enantiomer thereof, or a polymorph, pseudopolymorph or a prodrug thereof or pharmaceutically acceptable salt or hydrate thereof or a mixture thereof.

8. The composition of claim 7 wherein in the formula (I),
R₅ is CH₃, in S-orientation.
R₈ is 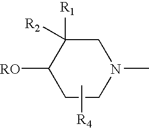
wherein:
R is hydrogen, C₁-C₆ alkyl, glycosyl, aralkyl , C₁-C₆ alkanoyl , or aminoalkanoyl or R is C₆H₁₁O₆, PO₃H₂ or SO₃H thus giving respectively the gluconic acid, phosphoric acid and sulfonic acid ester derivatives of the compounds;
R₁ and R₂ are the same or different and are selected from H, C₁₋₄ alkyl, aralkyl, aminoalkyl, trifluoroalkyl or halogen;
R₄ is H, C₁₋₄ alkyl, CF₃, phenyl, or F; R₄ is present at one or more of the positions of 2-, 4-, 5-, or 6- of the piperidine ring; and
R₁₀ is selected from H, C₁₋₅ alkyl, amino, alkylamino or acylamino groups.

9. The composition of claim 7, wherein the benzoquinolizine-2-carboxylic acid antimicrobial drug is selected from the group consisting of:
RS-(±)-9-fluoro-6,7-dihydro-8-(4-hydroxypiperidin-1-yl)-5-methyl-1-oxo-1H,5H-benzo[i,j]quinolizine-2-carboxylic acid;
R(+)-9-fluoro-6,7-dihydro-8-(4-hydroxypiperidin-1-yl)-5-methyl-1-oxo-1H,5H-benzo[i,j]quinolizine-2-carboxylic acid;
S-(−)-9-fluoro-6,7-dihydro-8-(4-hydroxypiperidin-1-yl)-5-methyl-1-oxo-1H,5H-benzo[i,j]quinolizine-2-carboxylic acid;
RS-(±)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof;
R(+)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof;
S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof;
RS-(±)-9-fluoro-6,7-dihydro-8-(4-hydroxypiperidin-1-yl)-5-methyl-1-oxo-1H,5H-benzo[i,j]quinolizine-2-carboxylic acid 0.2 hydrate;
R(+)-9-fluoro-6,7-dihydro-8-(4-hydroxypiperidin-1-yl)-5-methyl-1-oxo-1H,5H-benzo[i,j]quinolizine-2-carboxylic acid 0.2 hydrate;
S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate;
S-(−)-9-fluoro-6,7-dihydro-8-{trans-4-(RS)-hydroxy-3-(RS)-methylpiperidin-1-yl}-5-methyl-1-oxo-1H,5H-benzo[i,j]quinolizine-2-carboxylic acid;
S-(−)-9-fluoro-6,7-dihydro-8-{cis-4-(RS)-hydroxy-3-(RS)-methylpiperidin-1-yl-5-methyl-oxo-1H,5H-benzo[i,j]quinolizine-2-carboxylic acid;
S-(−)-9-fluoro-6,7-dihydro-8-{cis-(−)-4-R-hydroxy-3-S-methylpiperidin-1-yl}-5-methyl-1-oxo-1H,5H-benzo[i,j]quinolizine-2-carboxylic acid;
S-(−)-9-fluoro-6,7-dihydro-8-{cis-(+)-4-S-hydroxy-3-R-methylpiperidin-1-yl}-5-methyl-1-oxo-1H,5H-benzo[i,j]quinolizine-2-carboxylic acid; and
S-(−)-9-fluoro-6,7-dihydro-8-(3-ethyl-4-hydroxypiperidin-1-yl)-5-methyl-1-oxo-1H,5H-benzo[i,j]quinolizine-2-carboxylic acid (mixture of cis racemate and trans racemate) and pure stereoisomers thereof.

10. The composition of claim 9, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, or a solvatomorphic or polymorphic form thereof.

11. The composition of claim 9, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate.

12. The composition of claim 9, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid.

13. The composition of claim 1, wherein the benzoquinolizine-2-carboxylic acid antimicrobial drug comprises about 0.1% to about 1.0% by weight of the composition.

14. The composition of claim 1, wherein the amino acid is selected from arginine, histidine, arginine acetate, arginine-glutamate, arginine monohydrochloride, histidine acetate, histidine acetate dihydrate, histidine monohydrochloride, histidine monohydrochloride monohydrate, lysine, lysine acetate, lysine monohydrochloride, ornithine, tryptophan or salts thereof.

15. The composition of claim 14, wherein the amino acid comprises L-arginine.

16. The composition of claim 14, wherein the amino acid comprises L-lysine.

17. The composition of claim 1, wherein the cyclodextrin polymer is selected from α-cyclodextrin, β-cyclodextrin, γ-cyclodextrin, hydroxypropyl β-cyclodextrin or derivatives thereof.

18. The composition of claim 17, wherein the cyclodextrin polymer comprises hydroxypropyl β-cyclodextrin.

19. The composition of claim 1, wherein the solubilizing agent comprises about 1.5% to about 3.5% by weight of the composition.

20. The composition of claim 14, wherein the solubilizing agent is amino acid and comprises about 0.1% to about 1.4% by weight of the composition.

21. The composition of claim 17, wherein the solubilizing agent is cyclodextrin polymer and comprises about 1.5% to about 3.5% by weight of the composition.

22. The composition of claim 1, further comprising a pharmaceutically acceptable vehicle comprising a modifying agent selected from acids, bases, inorganic basic salts, organic basic salts, buffering agents or mixtures thereof and/or an agent for adjusting osmolality in amounts whereby the solution is substantially isotonic and has a physiologically acceptable pH.

23. The composition of claim 1, that is in a physical form selected from a concentrate, lyophilisate, powder, solution, or suspension.

24. A method of treating and/or preventing a bacterial infection disease in a subject comprising administering to the subject, a pharmaceutical composition of claim 1 in a therapeutically or prophylactically effective dose.

25. The method of claim 24, wherein the composition is diluted in a pharmaceutically acceptable liquid prior to being administered to the subject.

26. The method of claim 24, wherein the subject is a human or animal subject.

27. The method of claim 24, wherein the benzoquinolizine-2-carboxylic acid antimicrobial drug is selected from S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, or solvatomorphic or polymorphic forms thereof;
S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate; or
S-(−)-9-fluoro-6,7-dihydro-8-(4-hydroxypiperidin-1-yl)-5-methyl-1-oxo-1H,5H-benzo[i,j]quinolizine-2-carboxylic acid.

28. The method of claim 24, wherein the daily dose is about 0.01 mg to 100 mg/kg of S-(−)-9-fluoro-6,7-dihydro-8-(4-hydroxypiperidin-1-yl)-5-methyl-1-oxo-1H,5H-benzo[i,j]quinolizine-2-carboxylic acid, its arginine salt or 0.2 hydrate thereof.

29. The method of claim 24, wherein the benzoquinolizine-2-carboxylic acid antimicrobial drug comprises about 0.1 to 10% by weight of the composition.

30. The method of claim 24, wherein said solubilizing agent is selected from the group consisting of amino acids, cyclodextrin polymers or their derivatives, or mixtures thereof.

31. The method of claim 24, wherein said composition is administered by intravenous injection or infusion.

32. The method of claim 24, wherein the route of administration is parenteral.

33. A process for preparing a pharmaceutical composition comprising: mixing a pharmaceutically effective amount of benzoquinolizine-2-carboxylic acid antimicrobial drug of the formula (I) according to claim 1 with a pharmaceutically acceptable vehicle comprising a solubilizing agent at a concentration effective to maintain the drug in solution at physiologically compatible pH.

34. The process of claim 33, wherein said solubilizing agent is selected from amino acids, cyclodextrin polymers or their derivatives, or mixtures thereof.

35. The process of claim 34, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, solvatomorphic or polymorphic forms thereof; S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate, or S-(−)-9-fluoro-6,7-dihydro-8-(4-hydroxypiperidin-1-yl)-5-methyl-1-oxo-1H,5H-benzo[i,j]quinolizine-2-carboxylic acid.
